(19) United States
(12) Patent Application Publication
(10) Pub. No.: US 2009/0118229 A1
(43) Pub. Date: May 7, 2009

(54) CAROTENOID-CONTAINING COMPOSITIONS AND METHODS

(75) Inventors: Zeina Jouni, Evansville, IN (US); Zeina Makhoul, Tucson, AZ (US)

Correspondence Address: BRISTOL-MYERS SQUIBB COMPANY - MEAD JOHNSON, 2400 WEST LLOYD EXPRESSWAY, PATENT DEPARTMENT, EVANSVILLE, IN, 47721, US

(73) Assignee: Bristol-Myers Squibb Company

(21) Appl. No.: 12/206,856

(22) Filed: Sep. 9, 2008

Related U.S. Application Data
(60) Provisional application No. 60/986,119, filed on Nov. 7, 2007.

(51) Int. Cl. A61K 31/715; A61K 31/05; A61K 31/202; A61P 3/04; A61P 11/00; A61P 19/00

(52) U.S. Cl. 514/54; 514/733; 514/560

(57) ABSTRACT

The present invention is directed to carotenoid-containing compositions and methods for improving bone or respiratory health in a subject comprising administering to the subject a combination of lycopene, beta-carotene, and beta-cryptoxanthin.

### CROSS-REFERENCE TO RELATED PATENT APPLICATIONS

This application claims the priority benefit of U.S. Provisional Application No. 60/986,119, filed Nov. 7, 2007, which is incorporated by reference herein in its entirety.

### BACKGROUND OF THE INVENTION

(1) Field of the Invention

The present invention relates generally to carotenoid-containing compositions and methods for improving bone or respiratory health in a subject through administration of carotenoids.

(2) Description of the Related Art

Bone health is critically important to the overall health and quality of life of human beings. Healthy bones provide the body with a frame that allows for mobility and for protection against injury. Bones also serve as a storehouse for minerals that are vital to the functioning of many other life-sustaining systems in the body. Unhealthy bones, however, perform poorly in executing these functions. Unhealthy bones also lead to fractures, which are by far the most important consequence of poor bone health because they can result in disability, diminished function, loss of independence, and premature death.

An estimated 1.5 million individuals suffer a bone disease-related fracture each year. Fractures can have devastating consequences for both the individuals who suffer them and their family members. For example, hip fractures are associated with increased risk of mortality. The risk of mortality is between 2.8 and 4 times greater among hip fracture patients during the first 3 months after the fracture, as compared to the comparable risk among individuals of similar age that do not suffer a fracture. Those who are in poor health or living in a nursing home at the time of fracture are particularly vulnerable. For those who do survive, these fractures often precipitate a downward spiral in physical and mental health that dramatically impairs quality of life. Nearly one in five hip fracture patients, for example, ends up in a nursing home, a situation that a majority of participants in one study compared unfavorably to death. Many fracture victims become isolated and depressed, as the fear of falls and additional fractures mentally paralyzes them. Spine fractures, which are not as easily diagnosed and treated as fractures at other sites, can become a source of chronic pain as well as disfigurement.

Osteoporosis is the primary cause of fractures in the elderly. Although osteoporosis can be defined as low bone mass leading to structural fragility, it is difficult to determine the extent of the condition described in these qualitative terms, Using the World Health Organization's quantitative definition based on bone density measurement, there are roughly 10 million Americans over age 50 with osteoporosis and an additional 34 million with low bone mass or “osteopenia” of the hip, which puts them at risk for osteoporosis, fractures, and their potential complications later in life. In addition to fractures, bone diseases such as Paget's disease of the bone, osteogenesis imperfecta, rickets, osteomalacia, renal osteodystrophy, and hyperparathyroidism all remain major public health problems in this country.

Tremendous progress has been made in bone health in the last several decades, particularly in the past 15 years. Physical activity and calcium and vitamin D intake are now known to be major contributors to bone health for individuals of all ages. Advances in knowledge about risk factors have allowed work to begin on tools that assess the potential for bone disease in an individual. These risk-factor assessment tools help to identify high-risk individuals in need of further evaluation.

Even though bone disease often strikes late in life, the importance of beginning prevention at a very young age and continuing it throughout life is now well understood. During childhood and adolescence, bone mass acquisition occurs primarily through skeletal growth. It is generally accepted that bone mass acquisition throughout childhood is an important determinant of the risk of osteoporosis and other bone diseases in late adulthood. Therefore, a need exists for a method for improving bone health in a subject.

Poor respiratory health can be reflected through respiratory allergies or illnesses. For example, asthma is a respiratory allergy characterized by development of airway hyperreactivity, breathlessness, wheezing on exhale, dry cough and a feeling of tightness in the chest. Repeated allergen exposure can sustain the inflammatory immune response in the airways, resulting in a remodeling of the airways, commonly known as chronic asthma. Respiratory allergies in children are likely to appear in the form of asthma.

The prevention of respiratory allergies is especially important in infants and young children, as it appears that early allergic sensitization to allergens is associated with a delay in the maturation of normal immune responses. Not only is there a strong association between allergic sensitization and asthma, but the association appears to be age-dependent. Although few children become allergen sensitized during the first few years of life, the great majority of those who do become sensitized during this period develop asthma-like symptoms later in life. Martinez, F., Viruses and Atopic Sensitization in the First Years of Life, Am. J. Respir. Crit. Care Med., 162:S95-S99 (2000). Thus, it is important to find ways to prevent early allergen sensitization, prevent respiratory allergies later in life, and improve overall respiratory health.

Similarly, poor respiratory health can result in respiratory tract infections, which are extremely common in infants. In the first year of life, infants are prone to recurrent respiratory tract infections, often experiencing between three and six infections during that year alone.

Most upper and lower respiratory infections are caused by viruses for which no specific prevention or treatment is currently available. Some respiratory infections, including influenza, may be prevented with a vaccination. However, even when vaccinations are developed for specific respiratory infections, they are expensive and not universally available. Similarly, drugs to treat these infections have limited availability and are expensive. Traditional medications for respiratory allergies include antihistamines, topical nasal steroids, decongestants, and cromolyn solution, which may be equally expensive and not universally available. Thus, it would be useful to provide a non-medicinal method for the treatment or prevention of respiratory infections or allergies and the improvement of overall respiratory health.

### SUMMARY OF THE INVENTION

Briefly, therefore, the present invention is directed, in an embodiment, to a nutritional composition comprising lycopene, beta-carotene, beta-cryptoxanthin, galacto-oligosaccharide, and polydextrose.

In another embodiment, the invention is directed to a method for improving bone health in a subject comprising administering to the subject a combination of lycopene, beta-carotene, and beta-cryptoxanthin.

The invention is also directed, in an embodiment, to a method for improving respiratory health comprising administering to the subject a combination of lycopene, beta-carotene, and beta-cryptoxanthin.

In yet another embodiment, the invention is directed to a method for preventing obesity in a subject comprising administering to the subject a combination of lycopene, beta-carotene, and beta-cryptoxanthin.

### DETAILED DESCRIPTION OF THE PREFERRED EMBODIMENTS

Reference now will be made in detail to the embodiments of the invention, one or more examples of which are set forth below. Each example is provided by way of explanation of the invention, not a limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment.

Thus, it is intended that the present invention covers such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

Carotenoids are a related group of greater than 600 natural compounds, irrespective of geometric and stereoisomers, with demonstrated antioxidant efficacy. The carotenoids are broadly divided into “carotenes,” or non-oxygen substituted hydrocarbon carotenoids, and “xanthophylls,” oxygen-substituted carotenoids. Between 500 and 600 carotenoids have been identified, of which only about 24 occur in human foodstuffs. The major carotenoids found in foods are α-carotene, ⊕-carotene, lycopene, lutein, zeaxanthin, and β-cryptoxanthin. They are present in foods such as carrots, pumpkins, sweet potatoes, tomatoes, and other deep green, yellow, orange, red fruits and vegetables. Most carotenoids occur in nature predominantly in the all-trans form. Three of these carotenoids, α-carotene, β-carotene and β-cryptoxanthin, can be converted into retinol and are therefore considered provitamin A carotenoids. Lycopene, lutein and zeaxanthin do not have a vitamin A function and are referred to as nonprovitamin A carotenoids.

An important feature of carotenoids is a centrally located, extended conjugated double-bond system, which is responsible for the chemical reactivity, light-absorbing properties, and, thus, color of carotenoids. Potential biological function is determined by the chemical structure of carotenoids. The alternating single and double bond of the polyene backbone of carotenoids allow them to absorb excess energy from other molecules, which accounts for their antioxidant properties. They perform their antioxidant function by either quenching singlet oxygen and/or blocking free radical-mediated reactions. The polarity of the specific end groups of carotenoids accounts for the differences in how they interact with biological membranes. Carotenoids are associated with lipid portions of human tissues, cells, and membranes and bind to hydrophobic surfaces because they are lipophilic. In addition, carotenoids are easily isomerized and oxidized due to their high unsaturation and hence may lose biological activity after processing and storage.

In recent years, carotenoids have received the interest of researchers from diverse fields including food science, pharmacy, biochemistry and nutrition because of their wide spectrum of biological functions such as provitamin A, antioxidant, immuno-enhancement, and prevention of degenerative diseases.

For example, see U.S. Pat. No. 6,589,579 to Canan-Calvo, et al., U.S. Patent App. Pub. No. 2006/0106115 to Yamaguchi, U.S. Patent App. Pub. No. 2007/0015237 to Bailey, et al., or U.S. Patent App. Pub. No. 2007/0118916 to Puzio, et al.

The technical problem to be solved by the present invention is to provide novel carotenoid nutritional compositions that are useful in improving bone and/or respiratory health in subjects. Thus, in an embodiment, the present invention is directed to a nutritional composition comprising lycopene, beta-carotene, beta-cryptoxanthin, galacto-oligosaccharide, and polydextrose. The present invention is also directed, in an embodiment, to a method for improving bone or respiratory health by administering to them an effective amount of lycopene, beta-carotene, and beta-cryptoxanthin.

Lycopene, similar to other carotenoids, is a natural fat-soluble red pigment and photochemical found in certain plants such as tomatoes, watermelon, papaya, pink grapefruit and pink guava. Lycopene may protect humans against certain disorders, such as cancer and coronary heart disease.

Lycopene is an acyclic isomer of beta-carotene. Lycopene is a 40 carbon atom, open chain polyisoprenoid with 11 conjugated double bonds. Lycopene is a terpene assembled from 8 isoprene units. The color of lycopene is due to its many conjugated carbon double bonds. Each double bond reduces the energy required for electrons to transition to higher energy states, allowing the molecule to absorb visible light of progressively longer wavelengths. Lycopene absorbs most of the visible spectrum, so it appears red.

The structural formula of lycopene is as follows:

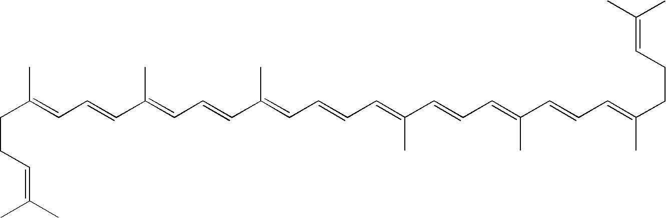

In an embodiment of the invention, the effective amount of lycopene is within the range of about 0.01 mg and about 10 mg per kg body weight per day. In another embodiment of the invention, the effective amount of lycopene is within the range of about 0.1 mg and about 5 mg per kg body weight per day. In a particular embodiment of the invention the effective amount of lycopene is about I mg per kg body weight per day.

Like lycopene, beta-carotene is a carotenoid. Beta-carotene is the most common of the carotenes and can be found in yellow, orange, and green leafy fruits and vegetables. It is unclear whether beta-carotene has any biological function for humans other than as a precursor for vitamin A. There is some evidence that beta-carotene may play a beneficial role in human nutrition beyond its provitamin A function. Beta-carotene has antioxidant activity, at least in vitro, and it may enhance intercellular communication and may have immunomodulatory and anticarcinogenic activities in certain circumstances.

The structure of beta-carotene is set forth below:

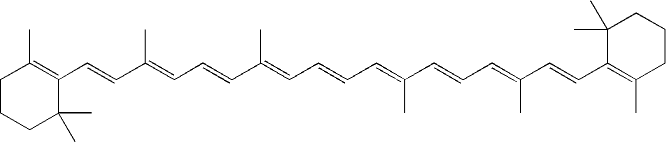

In an embodiment of the invention, the effective amount of beta-carotene is within the range of about 0.01 mg and about 10 mg per kg body weight per day. In another embodiment of the invention, the effective amount of beta-carotene is within the range of about 0.1 mg and about 5 mg per kg body weight per day. In a particular embodiment of the invention, the effective amount of beta-carotene is about 1 mg per kg body weight per day.

Beta-cryptoxanthin is a carotenoid that is found in oranges, papayas, peaches, tangerines, the petals and flowers of plants in the genus Physalis, egg yolk, butter, and bovine blood serum. Structurally, cryptoxanthin is closely related to beta-carotene, with only the addition of a hydroxyl group. It is a member of the class of carotenoids known as xanthophylls.

In the human body, cryptoxanthin is converted to vitamin A (retinol) and is therefore considered a provitamin A. As with other carotenoids, cryptoxanthin is an antioxidant and may help prevent free radical damage to cells and DNA. Recent findings of an inverse association between beta-cryptoxanthin and lung cancer risk in several observational epidemiological studies suggest that beta-cryptoxanthin could potentially act as a chemopreventive agent against lung cancer.

The structure of beta-cryptoxanthin is set forth below.

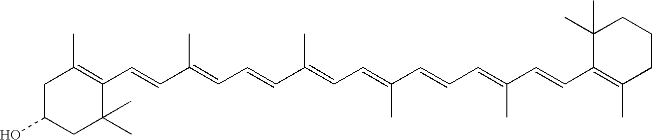

In an embodiment of the invention, the effective amount of beta-cryptoxanthin is within the range of about 0.01 mg and about 10 mg per kg body weight per day. In another embodiment of the invention, the effective amount of beta-cryptoxanthin is within the range of about 0.1 mg and about 5 mg per kg body weight per day. In a particular embodiment of the invention, the effective amount of beta-cryptoxanthin is about 1 mg per kg body weight per day.

As noted, the present invention is directed to a method for improving bone and respiratory health in subjects by administering to them an effective amount of lycopene, beta-carotene, and beta-cryptoxanthin. Some of the bone health benefits encompassed by the present invention include an increase of bone mineral content and bone mineral density, both of which are contributors to or are involved in bone development and growth; a decrease in osteoporosis; a decrease in the formation of osteoclasts (bone-reabsorbing cells) and/or an inducement of apoptosis of osteoclasts; and/or an increase in bone formation via an increase in alkaline phosphatase (enzyme involved in bone formation), increase in calcium absorption, increase in bone calcium content, increase in bone-DNA content (index of number of bone formation), increase in hormones involved in bone formation (such as osteoprotogerin, osteopontin, osteocalcin, IGF-1), increase in collagen, increase in transcription factors involved in bone formation (such as Runx-2), and/or increase of osteoblasts (bone forming cells).

Some of the respiratory benefits encompassed by the present invention include prevention or treatment of asthma; increased oxygen diffusivity in the blood and lungs; a reduction of local hypoxia at the blood-tissue interface of the blood vessel walls; an increased content of carotenoids in the lung tissue; an increased Forced Expiratory Volume in 1 second (FEV1) and Forced Vital Capacity (FVC); and/or a decrease in the decline in FEV1.

As used in the present invention, the source of the lycopene, beta-carotene, or beta-cryptoxanthin can be any source known in the art such as plant material, seafood, and/or single cell. In certain embodiments, one or more of the carotenoids may be in raw form or may be chemically manipulated. In a particular embodiment, one or more of the carotenoids may be genetically modified organisms.

In an embodiment, the lycopene, beta-carotene, and beta-cryptoxanthin may be administered in the form of a nutritional composition, infant formula, human milk supplement, or children's nutritional product. As used herein, the term “infant formula” means a composition that satisfies the nutrient requirements of an infant by being a substitute for human milk. Thus, the method of the invention is useful in preventing or treating bacterial infections in human infants, children, or adults.

If the lycopene, beta-carotene, and beta-cryptoxanthin are administered via an infant formula, the infant formula may be nutritionally complete and contain suitable types and amounts of lipid, carbohydrate, protein, vitamins and minerals. The amount of lipid or fat typically can vary from about 3 to about 7 g/100 kcal. The amount of protein typically can vary from about 1 to about 5 g/100 kcal. The amount of carbohydrate typically can vary from about 8 to about 12 g/100 kcal. Protein sources can be any used in the art, e.g., nonfat milk, whey protein, casein, soy protein, hydrolyzed protein, and/or amino acids. Carbohydrate sources can be any used in the art, e.g., lactose, glucose, corn syrup solids, maltodextrins, sucrose, starch, and/or rice syrup solids. Lipid sources can be any used in the art, e.g., vegetable oils such as palm oil, canola oil, corn oil, soybean oil, palmolein, coconut oil, medium chain triglyceride oil, high oleic sunflower oil, and/or high oleic safflower oil.

Conveniently, commercially available nutritional compositions, infant formulas, human milk supplements, or children's nutritional products can be used. For example, Enfalac, Enfamil®, Enfamil® Premature Formula, Enfamil® with Iron, Enfamil® LIPIL®, Lactofree®, Nutramigen®, Pregestimil®, and ProSobee®) (available from Mead Johnson & Company, Evansville, Ind., U.S.A.) may be supplemented with suitable levels of lycopene, beta-carotene, and beta-cryptoxanthin and used in practice of the method of the invention.

If the lycopene, beta-carotene, and beta-cryptoxanthin are administered in an infant formula, the amounts of each carotenoid in the formula may be up to about 40 nmol/g fat. In another embodiment, the amounts of each carotenoid in the formula may be within the range of about 2 nmol/g and about 35 nmol/g fat. In a particular embodiment, the amounts of each carotenoid in the formula may be within the range of about 5 nmol/g and about 30 nmol/g fat.

In other embodiments, if the lycopene, beta-carotene, and beta-cryptoxanthin are administered in an infant formula, the amounts of each carotenoid in the formula may be within the range of about 0.01 ppm and about 20 ppm carotenoid by weight of the total lipid content. In another embodiment, the amounts of each carotenoid in the formula may be within the range of about 0.1 ppm and about 10 ppm carotenoid by weight of the total lipid content.

The total carotenoid blend may comprise, in an embodiment, up to about 2000 mcg/L infant formula. In other embodiments, the total carotenoid blend may comprise from about 150 mcg/L to about 1500 mcg/L infant formula. In yet another embodiment, the total carotenoid blend may comprise from about 200 mcg/L to about 1200 mcg/L infant formula.

The individual carotenoids may be present in the infant formula in an amount of from about 50 mcg/L to about 1150 mcg/L, about 75 mcg/L to about 230 mcg/L, or about 100 mcg/L to about 200 mcg/L.

In some embodiments of the invention, additional components may be administered in combination with lycopene, beta-carotene, and beta-cryptoxanthin. These additional components may include probiotics, prebiotics, or long chain polyunsaturated fatty acids (LCPUFAs). The components may be administered separately from the lycopene, beta-carotene, and beta-cryptoxanthin or may be included as part of a nutritional composition, infant formula, human milk supplement, or children's nutritional product that contains lycopene, beta-carotene, beta-cryptoxanthin, and one or more additional components.

The term “probiotic” means a microorganism that exerts beneficial effects on the health of the host. Any probiotic known in the art may be used, provided it is suitable for combination with the other components of the supplement. For example, the probiotic may be chosen from the group consisting of Lactobacillus and Bifidobacterium. Alternatively, the probiotic can be Lactobacillus rhamnosus GG.

The term “prebiotic”, as used herein, means a non-digestible food ingredient that stimulates the growth and/or activity of probiotics. In this embodiment, any prebiotic known in the art may be used, provided it is suitable for combination with the other components of the supplement. In a particular embodiment, the prebiotic can be selected from the group consisting of fructo-oligosaccharide, gluco-oligosaccharide, galacto-oligosaccharide, insulin, isomalto-oligosaccharide, polydextrose, xylo-oligosaccharide, lactulose, and combinations thereof. In a particular embodiment, the prebiobic is a mixture of galacto-oligosaccharide and polydextrose.

In an embodiment, the total amount of prebiotics present in the nutritional composition may be from about 1.0 g/L to about 10.0 g/L of the composition. In another embodiment, the total amount of prebiotics present in the nutritional composition may be from about 2.0 g/L and about 8.0 g/L of the composition. In yet another embodiment, the total amount of prebiotics present in the nutritional composition may be about 4.0 g/L of the composition.

If galacto-oligosaccharide is used as a prebiotic, the amount of galacto-oligosaccharide in the nutritional composition may, in an embodiment, be within the range of from about 1.0 g/L to about 4.0 g/L. In another embodiment, the amount of galacto-oligosaccharide in the nutritional composition may be about 2.0 g/L. If polydextrose is used as a prebiotic, the amount of polydextrose in the nutritional composition may, in an embodiment, be within the range of from about 1.0 g/L to about 4.0 g/L. In another embodiment, the amount of polydextrose in the nutritional composition may be about 2.0 g/L. In a particular embodiment, galacto-oligosaccharide and polydextrose are supplemented into the nutritional composition in a total amount of about 4.0 g/L. In this embodiment, the amount of galacto-oligosaccharide may be about 2.0 g/L and the amount of polydextrose may be about 2.0 g/L.

While not wishing to be bound to this or any theory, it is believed that the administration of prebiotics and carotenoids may provide a synergistic effect. More specifically, it is believed that the prebiotic may increase the absorption of calcium and other minerals, further enhancing the bone health benefits discussed in the present application.

In yet another embodiment of the invention, LCPUFAs may be administered in combination with lycopene, beta-carotene, and beta-cryptoxanthin. In this embodiment, the LCPUFAs may include docosahexaenoic acid (DHA), arachidonic acid (ARA), and/or eicosapentaenoic acid (EPA).

If administered as part of the present invention, the weight ratio of ARA:DHA may be from about 1:3 to about 9:1. In one embodiment of the present invention, this ratio is from about 1:2 to about 4:1. In yet another embodiment, the ratio is from about 2:3 to about 2:1. in one particular embodiment the ratio is about 2:1. In another particular embodiment of the invention, the ratio is about 1:1.5. in other embodiments, the ratio is about 1:1.3. In still other embodiments, the ratio is about 1:1.9. In a particular embodiment, the ratio is about 1.5:1. In a further embodiment, the ratio is about 1.47:1.

If administered as part of the present invention, the level of DHA may be within the range of about 0.0% and about 1.00% of fatty acids, by weight. In other embodiments, the level of DHA may be about 0.32% by weight. In some embodiments, the level of DHA may be about 0.33% by weight. In another embodiment, the level of DHA may be about 0.64% by weight. In another embodiment, the level of DHA may be about 0.67% by weight. In yet another embodiment, the level of DHA may be about 0.96% by weight. In a further embodiment, the level of DHA may be about 1.00% by weight.

If administered as part of the present invention, the level of ARA may be within the range of about 0.0% and about 0.67% of fatty acids, by weight. In another embodiment, the level of ARA may be about 0.67% by weight. In another embodiment, the level of ARA may be about 0.5% by weight. In yet another embodiment, the level of DHA may be within the range of about 0.47% and about 0.48% by weight.

If administered as part of the present invention, the amount of DHA may be from about 2 mg/100 kilocalories (kcal) to about 100 mg/100 kcal. In another embodiment, the amount of DHA may be from about 5 mg/100 kcal to about 75 mg/100 kcal. In yet another embodiment, the amount of DHA may be from about 15 mg/100 kcal to about 60 mg/100 kcal.

If administered as part of the present invention, the amount of ARA may be from about 4 mg/100 kilocalories (kcal) to about 100 mg/100 kcal. In another embodiment, the amount of ARA may be from about 10 mg/100 kcal to about 67 mg/100 kcal. In yet another embodiment, the amount of ARA may be from about 20 mg/100 kcal to about 50 mg/100 kcal. In a particular embodiment, the amount of ARA may be from about 25 mg/100 kcal to about 40 mg/100 kcal. In one embodiment, the amount of ARA is about 30 mg/100 kcal.

If administered as part of the present invention, the effective amount of DHA may be from about 3 mg per kg of body weight per day to about 150 mg per kg of body weight per day. In one embodiment of the invention, the amount is from about 6 mg per kg of body weight per day to about 100 mg per kg of body weight per day. In another embodiment the amount is from about 15 mg per kg of body weight per day to about 60 mg per kg of body weight per day.

If administered as part of the present invention, the effective amount of ARA may be from about 5 mg per kg of body weight per day to about 150 mg per kg of body weight per day. In one embodiment of this invention, the amount varies from about 10 mg per kg of body weight per day to about 120 mg per kg of body weight per day. In another embodiment, the amount varies from about 15 mg per kg of body weight per day to about 90 mg per kg of body weight per day. In yet another embodiment, the amount varies from about 20 mg per kg of body weight per day to about 60 mg per kg of body weight per day.

If the composition of the invention is supplemented with oils containing LCPUFAs, it may be accomplished using standard techniques known in the art. For example, an equivalent amount of an oil which is normally present in a composition, such as high oleic sunflower oil, may be replaced with the LCPUFAs.

If utilized, the source of the LCPUFAs can be any source known in the art such as marine oil, fish oil, single cell oil, egg yolk lipid, and/or brain lipid. The LCPUFAs can be in natural form or refined form.

In other embodiments of the invention, lycopene, beta-carotene, and beta-cryptoxanthin may be combined and administered to a subject for the purpose of treating or preventing any of the following: reflux, spitting up, abdominal pain, bloating, vomiting, gastric inflammation, gastritis, ulcer formation, hypertension, dyslipidemia, Type I and II diabetes, insulin sensitivity, obesity, cardiovascular disease, cancer, atherosclerosis. In other embodiments, lycopene, beta-carotene, and beta-cryptoxanthin can be combined and administered for the purpose of improving digestion or stool consistency, modulating antioxidant enzymes, decreasing cellular and tissue oxidative stress, shifting T-helper cell Types 1 to Th2 balance, and modulating immune function.

In some embodiments, the invention includes a method for improving weight management in a subject comprising administering to the subject an effective amount of lycopene, beta-carotene, and beta-cryptoxanthin. In other embodiments, the invention includes a method for preventing or treating obesity in a subject comprising administering to the subject an effective amount of lycopene, beta-carotene, and beta-cryptoxanthin. Obesity has been linked with an inflammation of adipose tissue. In some studies, inflammation has also been identified as an early characteristic of obesity. The combination of lycopene, beta-carotene, and beta-cryptoxanthin, in addition to their antioxidant benefits, may contribute to a reduction in inflammation, thereby reducing or preventing the onset of obesity in the present invention.

In an embodiment, the invention is directed to the use of a combination of lycopene, beta-carotene, and beta-cryptoxanthin in the manufacture of an ingestible composition for improving bone health in a subject. In another embodiment, the invention is directed to use of a combination of lycopene, beta-carotene, and beta-cryptoxanthin in the manufacture of an ingestible composition for improving respiratory health in a subject. In yet another embodiment, the invention is directed to use of a combination of lycopene, beta-carotene, and beta-cryptoxanthin in the manufacture of an ingestible composition for reducing or preventing asthma in a subject. Additionally, in another embodiment, the invention is directed to use of a combination of lycopene, beta-carotene, and beta-cryptoxanthin in the manufacture of an ingestible composition for preventing obesity in a subject.

The invention, in a particular embodiment, is directed to a combination of lycopene, beta-carotene, and beta-cryptoxanthin for use in improving bone health in a subject. The invention is also directed, in an embodiment, to a combination of lycopene, beta-carotene, and beta-cryptoxanthin for use in improving respiratory health in a subject. The invention is additionally directed, in an embodiment, to a combination of lycopene, beta-carotene, and beta-cryptoxanthin for use in reducing or preventing asthma in a subject. In a further embodiment, the invention is directed to a combination of lycopene, beta-carotene, and beta-cryptoxanthin for use in preventing obesity in a subject.

All references cited in this specification, including without limitation, all papers, publications, patents, patent applications, presentations, texts, reports, manuscripts, brochures, books, internet postings, journal articles, and/or periodicals are hereby incorporated by reference into this specification in their entireties. The discussion of the references herein is intended merely to summarize the assertions made by their authors and no admission is made that any reference constitutes prior art. Applicants reserve the right to challenge the accuracy and pertinence of the cited references.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged in whole or in part. Furthermore, those of ordinary skill in the art will appreciate that the foregoing description is by way of example only, and is not intended to limit the invention so further described in such appended claims. Therefore, the spirit and scope of the appended claims should not be limited to the description of the preferred versions contained therein.

What is claimed is:

1. A nutritional composition comprising lycopene, beta-carotene, beta-cryptoxanthin, galacto-oligosaccharide, and polydextrose.

2. The nutritional composition of claim 1 wherein the nutritional composition contains each of lycopene, beta-carotene, and beta-cryptoxanthin in an amount within the range of from about 50 to about 1150 mcg/L.

3. The nutritional composition of claim 1 wherein the nutritional composition contains each of galacto-oligosaccharide and polydextrose in an amount within the range of from about 1.0 g/L to about 4.0 g/L.

4. The nutritional composition of claim 1 additionally comprising at least one long-chain polyunsaturated fatty acid selected from the group consisting of docosahexaenoic acid, arachidonic acid, eicosapentaenoic acid, and combinations thereof.

5. The nutritional composition of claim 1 wherein the nutritional composition is in a form selected from those consisting of an infant formula, human milk fortifier, and children's nutritional product.

6. The nutritional composition of claim 1 wherein the nutritional composition is in a form selected from those consisting of powder and liquid.

7. A method for improving bone health in a subject comprising administering to the subject a combination of lycopene, beta-carotene, and beta-cryptoxanthin.

8. The method of claim 7 wherein the improvement of bone health is selected from the group consisting of an increased bone mineral content, an increased bone mineral density, a decreased risk of osteoporosis, a decrease in the formation of osteoclasts, the induction of apoptosis of osteoclasts, and increased bone formation.

9. The method of claim 8 wherein the increased bone formation occurs via a method selected from the group consisting of increased alkaline phosphatase, increased calcium absorption, increased calcium content, increased bone DNA content, increased hormones in bone formation, increased collagen, increased transcription factors involved in bone formation, and increased osteoblasts.

10. The method of claim 7 wherein the amount of lycopene administered is within the range of about 0.01 mg and about 10 mg per kg body weight per day.

11. The method of claim 7 wherein the amount of beta-carotene administered is within the range of about 0.01 mg and about 10 mg per kg body weight per day.

12. The method of claim 7 wherein the amount of beta-cryptoxanthin administered is within the range of about 0.01 mg and about 10 mg per kg body weight per day.

13. The method of claim 7 wherein the amount of lycopene administered is within the range of about 0.1 mg and about 5 mg per kg body weight per day, the amount of beta-carotene administered is within the range of about 0.1 mg and about 5 mg per kg body weight per day, and the amount of beta-cryptoxanthin administered is within the range of about 0.1 mg and about 5 mg per kg body weight per day.

14. The method of claim 7 additionally comprising the administration of at least one LCPUFA selected from the group consisting of DHA, ARA, EPA, and combinations thereof.

15. The method of claim 7 additionally comprising the administration of galacto-oligosaccharide and polydextrose.

16. The method of claim 7 wherein the lycopene, beta-carotene, and beta-cryptoxanthin are administered in the form of an infant formula.

17. A method for improving respiratory health in a subject comprising administering to the subject a combination of lycopene, beta-carotene, and beta-cryptoxanthin.

18. The method of claim 17 wherein the improvement in respiratory health comprises one or more selected from the group consisting of oxygen diffusivity in the blood or lungs, reduced local hypoxia at the blood-tissue interface of blood vessel walls, increased FEV1, increased FVC, and decreased decline in FEV1.

19. The method of claim 17, wherein the improvement in respiratory health comprises reducing or preventing asthma.

20. A method for preventing obesity in a subject comprising administering to the subject a combination of tycopene, beta-carotene, and beta-cryptoxanthin.
